(19) United States
(12) Patent Application Publication
(10) Pub. No.: US 2008/0318248 A1
(43) Pub. Date: Dec. 25, 2008

(54) Compositions and Methods of Screening Apoproteins

(75) Inventors: Herbert Holden Thorp, JR., Carrboro, NC (US); Thomas V. O'Halloran, Chicago, IL (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Viamet Pharmaceuticals, Inc, Durham, NC (US)

(21) Appl. No.: 12/090,753

(22) PCT Filed: Oct. 20, 2006
(86) PCT No.: PCT/US06/41221

§ 371 Date: Sep. 2, 2008

Related U.S. Application Data
(60) Provisional application No. 60/728,840, filed on Oct. 20, 2005.

(51) Int. Cl. G01N 33/53; G01N 33/566; C40B 30/04; C12N 5/02

(52) U.S. Cl. 435/7.1; 436/501; 506/9; 435/375

(57) ABSTRACT

The present invention is directed to compositions and methods of screening apoproteins for the elucidation of modulators of metalation of apoproteins.

### FIELD OF THE INVENTION

The present invention is directed to compositions and methods of screening apoproteins for the elucidation of modulators of metalation of apoproteins.

### BACKGROUND OF THE INVENTION

Thirty percent of all known proteins are metalloproteins that interact closely with a metal ion. For example, every known enzymatic class contains metalloproteins. In general, the structure and function of metalloproteins are highly dependent on the presence of the metal ions, and metal ion concentration in cells is highly regulated. Important metal ions include those of iron, zinc, copper, manganese, magnesium, potassium, calcium, molybdenum, vanadium, tungsten and cobalt. Recent work has shown the presence of particular integral membrane proteins that function to pump metal ions into cells that provides exceptional control over intracellular metal ion concentrations. In addition, metalation pathways are being elucidated. For example, recent work has elucidated the presence of several metallochaparone proteins that guide and protect the metal ions through the cytoplasm and transfer the metals to specific partner proteins. In addition, recent work has also shown that the amount of “free” metal ions with cells is very low. See for example Finney et al., Science 30:931 (2003); Outten et al., Science 292:2488 (2001).

From a therapeutic perspective, there are a number of metalloproteins that are associated with disease states or conditions. For example, cardiovascular disease, cancer, erectile dysfunction and glaucoma have all been associated with metalloproteins (ACE, HDAC and farnesyltransferase, PDE-5 and carbonic anhydrase, respectively). In addition, several genetic diseases, including hemochromatosis, Menkes, Wilson and Lou Gerhig's disease, have been correlated to metalation disorders.

Phosphodiesterases (PDEs) are metalloenzymes that catalyze the degradation of the cyclic nucleotides, cyclic AMP and cyclic GMP, to the corresponding 5′ nucleotide monophosphates. To date, 11 PDE families have been identified. As these can be derived from multiple genes, many capable of generating a number of isoforms, there currently exists over 50 known PDE enzymes. These enzymes exist as homodimers and there is structural similarity between the different families. However, they differ in several respects like selectivity for cyclic nucleotides, sensitivity for inhibitors and activators, physiological roles and tissue distribution. There are a number of specific inhibitors with therapeutic value. Phosphodiesterase type 5 inhibitors (PDE5) inhibitors include sildenafil (Viagra), vardenafil (Levitra), and tadalafil (Cialis). The PDE 4 family has also been extensively investigated, as inhibitors of these enzymes are known to be both potent anti-depressants and anti-inflammatory agents. Members of both families are also expressed in cells of the immune system and considerable attention has been focused on the potential of selective inhibitors of PDE 3 and PDE 4, or in combination, for the treatment of asthma and other inflammatory diseases. See Clayton et al., Respiratory Research 2004, 5:4.

In addition, matrix metalloproteinases (MMPs) have been shown to be involved in a number of disease states. MMPs constitute a family of over 20 zinc containing proteolytic enzymes that play an integral role in the physiology of the extracellular matrix (ECM). MMPs play a major role in the degradation of the basement membrane and the remodeling of the ECM. Certain normal physiological processes such as fetal development, inflammatory cell migration, wound healing and angiogenesis depend on the controlled and concerted activity of the MMPs and the natural tissue inhibitors of metalloproteinases (TIMPs). In pathological processes, such as cancer, specific MMPs may be recruited to permit primary tumor growth and metastatic disease. Activation of these MMPs has been implicated in tissue invasiveness, metastases and angiogenesis. There are three distinct classes of MMPs by target: collagenases (MMP-1, MMP-8, and MMP-13), stromelysins (MMP-3, MMP-10, MMP-11), and gelatinases (MMP-2 and MMP-9). MMPs are secreted into the ECM in a proenzyme form, which requires activation by other enzymes. One class of activators is the enzyme membrane type MMP (MT-MMP). The MT-MMPs (MMP-14, MMP-15, MMP-16, MMP-17) have a transmembrane domain and are essential in activating pro-MMP.

Among the MMPs mentioned above, over-expression of certain MMPs (MMP-2 and MMP-9) have been implicate development and have been evaluated extensively in different tumors such colon, gastric, head and neck, prostate, and lung cancer. For example, studies shown that in colorectal cancer, both MMP-2 and MMP-9 (in both pro and active forms) are over-expressed in cancerous tissue when compared with the normal mucosa. Similar findings have been shown in gastric cancer and pancreatic cancer (Gress T. M., Muller-Pillasch F., Lerch, M. M., et al. Expression and in-situ localization of genes coding for extracellular matrix proteins and extracellular matrix degrading proteases in pancreatic cancer. Int. J. Cancer 1995, 62:407-413; Normura H, Sato H. Seiki M. et al. Expression of membrane type-matrix metalloproteinase in human gastric cancinomas. Cancer Res 1995; 55:3263-3266; Parsons S L, Watson S A, Brown P D, et al. Matrix metalloproteinases. Br. J. Surg. 1997, 87: 160-166). MMP-2 and MMP-9 over-expression also appear to correlate with tumor stage, tumor aggressiveness and poor prognosis for gastrointestinal, cervical, bladder and lung tumors (Nuovo G J, MacConnell P B, Simisir A, et al. Correlation of the in situ detection of polymerase chain reaction-amplified metalloproteinase complementary DNA and their inhibitors with prognosis in cervical carcinoma. (Cancer Res. 1995, 55:267-275; Davies B, Wasman J, Wasan H, et al.). Levels of matrix metalloproteinases in bladder cancer correlate with tumor grade and invasion. (Cancer Res. 1993, 53: 5365-5369; Brown P D, Bloxidge R E, Stuart N S, et al.). Association between expression of activated 72-kilodalton gelatinase and tumor spread in non-small cell lung carcinoma. (J. Natl. Cancer Inst. 1993, 85: 574-578). Although there is clear over-expression of MMP in certain tumors, there is variability of over-expression of MMP in different tumor types. For example, Fieberg, et al, studied expression patterns of MMP-2, MMP-3, and MMP-7 in 47 human tumor xenografts. Their results showed varying degrees of MMP-2 over-expression with 100% of soft tissue sarcomas, 84% of melanomas, 53% of testicular carcinoma and 26% of bladder cancers exhibiting MMP-2 over-expression (Fieberg H, Klostermeyer A, Schuler J B). Characterization of matrix metalloproteinases in 47 human tumor xenografts who high expression of MMP2 in melanomas and sarcomas (abstract no. 3058). Proceedings of the 90^{th} Annual Meeting of the American Association for Cancer Research 1999 Apr. 10-13, Philadelphia (Pa.)). There are several drugs in clinical trials that inhibit MMPs or MT-MMPs.

In addition, there are a wide variety of other metalloproteins that have been associated with disease. For example, recent studies have shown that beta amyloid, involved with Alzheimer's disease, is a metalloprotein containing zinc, copper and iron. The NS5A protein of Hepatitis C Virus is a Znmetalloprotein as well. J. Biol. Chem., Vol. 279, Issue 47, 48576-48587, Nov. 19, 2004. Cytochrome c oxidase is a macromolecular metalloprotein complex essential for the respiratory function of the cell, and mutations in this gene have been shown to correlate to severe pathology in human infants. J Bioenerg Biomembr. 2002 October; 34(5):381-8. Myelin basic protein (MBP), implicated in myelin compaction, has been shown to be a Zn-binding protein.

Similarly, a number of viral proteins are metalloenzymes, such as the HIV-1 protease, HIV-1 reverse transcriptase, HCV NS5a protein, etc.

Overall, the key importance and sheer number of metalloproteins dictate that new methods for screening for binding to and modulation of metalloprotein bioactivity are needed.

### BRIEF SUMMARY OF THE INVENTION

The present invention provides methods for screening for binding to metalloproteins comprising providing an apoprotein and contacting the apoprotein with a candidate agent to form a mixture. The binding of the candidate agent to the apoprotein is then determined. In some aspects, this is done by adding a metal binding component to the mixture and determining the bioactivity of the protein.

In a further aspect, the invention includes providing a metalyated protein and removing the metal to form the apoprotein.

In an additional aspect, the invention provides methods of inhibiting the bioactivity of a metalloprotein comprising contacting an apoprotein form of the metalloprotein with an inhibitor such that metal binding to the apoprotein form is decreased.

In a further aspect, the invention provides methods of treating a metalloprotein-associated disorder comprising contacting an apoprotein form of the metalloprotein with an inhibitor such that metal binding to the apoprotein form is decreased.

### BRIEF DESCRIPTION OF THE DRAWINGS

FIG. 1 is a schematic of various mechanisms of the present invention.

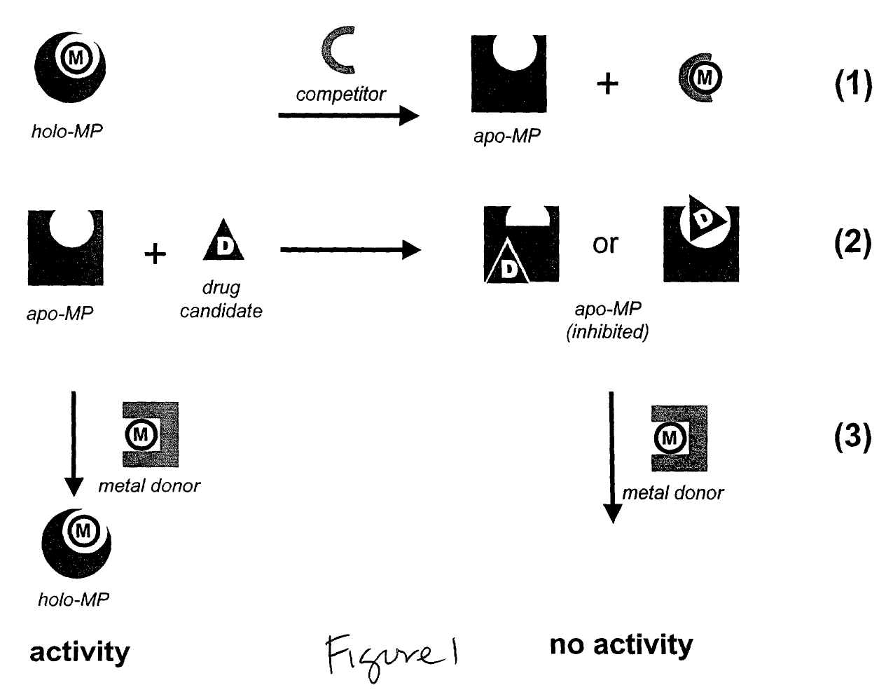

### DETAILED DESCRIPTION OF THE INVENTION

The present invention is directed to compositions and methods of screening for binding to and modulation of the activity of metalloproteins. Currently, it appears that metalloproteins correspond to roughly a third of all structurally characterized proteins. These proteins rely on metal ions for either bioactivity, such as the case with the matrix metalloproteins (MMPs), heme proteins and zinc finger proteins, as well as structural integrity, which can itself lead to activity without direct interaction of the metal in the activity. That is, metal ions may be important for correct structure, that enables bioactivity, rather than having the metal directly participate in the bioactivity, such as is the case for enzymatic activity in superoxide dismutase (SOD), for example. In addition, metalloproteins can contain both one or more bioactive metals as well as structural metals, and these metals can be the same or different. Thus for example SOD contains both Zn and Cu metals. Thus, the present invention relies on the fact that the presence of metal ions within many metalloproteins are crucial to activity.

Surprisingly, the amount of free metal ion within the cytoplasm of cells is quite low. See Changela, A., Chen, K., Xue, Y., Holschen, J., Outten, C. E., O'Halloran, T. V., Mondragon, A. “Molecular Basis of Selectivity and Zeptomolar Sensitivity by CueR.” Science, 2003, 301, 1383-1387. Finney, L. A., O'Halloran, T. V. “Transition Metal Speciation in the Cell: Insights from the Chemistry of Metal Ion Receptors.” Science, 2003, 300, 931-936. Outten, C. E., O'Halloran, T. V., “Femtomolar Sensitivity of Metalloregulatory Proteins Controlling Zinc Homeostasis.” Science, 2001, 292, 2488-2492. Rae, T. D., Schmidt, P. J., Pufahl, R. A., Culotta, V. C., and O'Halloran, T. V. “Undetectable Free Intracellular Copper: the Requirement of a Copper Chaperone for Superoxide Dismutase.” Science, 1999, 284, 805-808, all of which are expressly incorporated by reference herein.

As such, metalloproteins appear to “compete” with each other for metal ions, and many require particular metallochaperone proteins for metalation. Due to the fact that most, if not effectively all of the metal ions within a cell are associated with proteins, the present invention is directed to methods for screening for inhibitors of metalation; that is, the concentration of the apoprotein form of these metalloproteins may be quite significant within a cell, and thus affords the opportunity for drug agents to bind preferentially to the apo form of the metalloprotein, prevent metalation and thus decrease bioactivity.

Thus the invention provides methods that allow for screening of candidate agents that bind to metalloproteins to prevent metal activation of the bioactivity. This can be accomplished in several ways. In one embodiment, screening is done on apoproteins, e.g metalloproteins that are missing one or more metal ions (either by removal or via isolation prior to metalation). In this embodiment, candidate agents are screened for the ability to bind to the metalloprotein, and subsequently significantly prevent the addition of metal ions to the protein, such that the bioactivity of the protein is decreased or eliminated. Alternatively, screening can be done on metalated proteins for agents that remove the metal ion or alter the binding such that the bioactivity is reduced or eliminated.

Accordingly, the invention provides methods for screening for binding to the apoprotein form of metalloproteins. By “metalloprotein” herein is meant a protein that reversibly binds at least one metal ion. The metal ion is bound by one or more side chain or backbone atoms or by other binding functions that are bound to the protein; metalloproteins exhibit characteristic metal-to-protein stoichiometries that correspond to the minimal metal-protein ratio for a given structure or function; the structure and/or function of this type of protein may be dependent upon the metal being bound in a specific site of the protein. If metal-occupancy is required for a specific catalytic activity, the metalloproteins is called a metalloenzyme.

In general, metalloproteins (including apoproteins) from any organism can be used in the screening methods outlined herein, depending on the desired outcome. metalloproteins from any cell type may be used, including eukaryotic and prokaryotic cells.

Suitable metalloproteins come from eukaryotic cells including any animal, plant, yeast, fungal and protozoa cells. Suitable source eukaryotic cells include any animal, plant, yeast, fungal and protozoa cells. In some aspects, metalloproteins from eukaryotic cells are used, such as metalloproteins from mammalian cells, with rodent (e.g. mouse, rat, hamster, etc.) primate and human cells being particularly preferred. Accordingly, suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells, osteoclasts, chondrocytes and other connective tissue cells, keratinocytes, melanocytes, liver cells, kidney cells, adipocytes, pancreatic cells, islets of Langheran, neural cells (including immortalized neuoendocrine cells, neuroblastoma cells and glia cells), Schwanoma cell lines, organotypic or mixed cells in culture and beta cells . . . . Suitable cells also include known research cells, including, but not limited to, Jurkat T cells, NIH 3T3 cells, CHO, COS, etc. See the ATCC cell line catalog, hereby expressly incorporated by reference.

Suitable metalloproteins from bacteria include, but are not limited to, metalloproteins from pathogenic and non-pathogenic prokaryotes including Bacillus, including Bacillus anthracis; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. peffringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. Y. lamblia, Y. pestis, Pseudomonas, e.g. P. aeruginosa, P. putida; Chlamydia, e.g. C. trachomatis; Bordetella, e.g. B. pertussis; Treponema, e.g. T. palladium; B. anthracis, Y. pestis, Brucella spp., F. tularensis, B. mallei, B. pseudomallei, B. mallei, B. pseudomallei, C. botulinum, Salmonella spp., SEB V. cholerae toxin B, E. coli O157:H7, Listeria spp., Trichosporon beigelii, Rhodotorula species, Hansenula anomala, Enterobacter sp., Klebsiella sp., Listeria sp., Mycoplasma sp. and the like.

Mps from useful eukaryotic cells include metalloproteins from yeast cells, particularly Saccharomyces cerevisiae, Hansenula polymorpha, Kluyveromyces fragilis and K. lactis, Pichia guillerimondii and P. pastoris, Schizosaccharomyces pombe, plasmodium falciparium, and Yarrowia lipolytica.

Additional metalloproteins can be used from fungal cells including, but are not limited to, metalloproteins from Candida strains including Candida glabrata, Candida albicans, C. krusei, C. lusitaniae and C. maltosa, as well as species of Aspergillus, Cryptococcus, Histoplasma, Coccidioidesi, Blastomyces, Penicillium.

Additional metalloproteins can be used from protozoa, including, but are not limited to, metalloproteins from Trypanosoma, Leishmania species including Leishmania donovanii, Plasmodium spp., Pneumocystis carinii, Cryptosporidium parvum, Giardia lamblia, Entamoeba histolytica, and Cyclospora cayetanensis.

In one embodiment, the cells utilized in the assays are prokaryotic, for example to test for modulation of infectivity and activity based on metalation. For example, for some bacteria, virulence is associated with metal uptake, for example iron uptake. Thus, testing for modulation (e.g. inhibition) of metalation of bacterial proteins is provided.

In some aspects, the cells may be infected with viruses to test for agents that modulate viral apoproteins in infection and activity. metalloproteins from viruses can be used, including, but are not limited to, orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like. In this embodiment, it is possible to add the candidate agent prior to, simultaneously with, or after the introduction of the virus.

As indicated herein, the metal ion may directly contribute to bioactivity (e.g. as part of the catalytic mechanism) or function to confer structural integrity to allow bioactivity. “Bioactivity” in this context includes both enzymatic activity or activity based on protein binding (e.g. transcription factors), and will be determined based on the identity of the metalloprotein.

There are a wide number of suitable metalloproteins for use in the invention. In one embodiment, the metalloprotein is an enzyme, sometimes referred to herein as a metalloenzyme. Suitable classes of metalloenzymes include, but are not limited to, hydrolases such as proteases, carbohydrases, lipases and nucleases; isomerases such as racemases, epimerases, tautomerases, or mutases; transferases, kinases and phophatases, including phosphodiesterases.

In some embodiments, the metalloproteins are not enzymes, but have bioactivity based on other protein characteristics. Zn finger proteins are an example.

Particular metalloproteins of use in the invention include, but are not limited to, matrix metalloproteinases (including MT-MMPs) such as MMP-1, MMP-2, MMP-3, MMP-4, MMP-5, MMP-6, MMP-7, MMP-8, MMP-9, MMP-10, MMP-11, MMP-12, MMP-13, MMP-14, MMP-15, MMP-16 and MMP-17, myelin basic protein (MBP), NS5A of HCV, cytochrome c oxidase, VAP-1, SOD, anthrax lethal factor, 5-lipoxygenase, COX-2, VAP-1, PDF, 4-hydroxyphenylpyruvate dioxygenase, alcohol dehydrogenase, angiotensin converting enzyme (ACE), aromatase, metallo-beta-lactamase (bacterial), carbamoyl phosphate synthetase, carbonic anhydrase II, carbonic anhydrase I, catechol o-methyl-transferase, cyclooxygenase (non-selective), cyclooxygenase 2, cytochrome P450, DNA polymerase, EGFR tyrosine kinase, farnesyl diphosphate synthase, fumarate reductase (mitochondrial), heme oxygenase, HIV-1 reverse transcriptase, HIV-1 reverse transcriptase, lanosterol demethylase (fungal), neutral endopeptidase, PDE (non-selective), PDE III selective, PDE IV selective, PDE V selective, pyruvate:ferredoxin oxidoreductase, renal dipeptidase, ribonucleoside diphosphate reductase, RNA polymerase (bacterial), thyroid peroxidase, and xanthine oxidase.

Iron dependent proteins include, but are not limited to, heme proteins including COX-2, alpha keto glutarate dependent enzymes which require a non-heme iron cofactor; ribonucleotide reductase (many forms have two non-heme irons in the active site), peptide deformylase, anthrax lethal factor, etc.

As outlined herein, metalloproteins contain one or more metal ions, including, but not limited to, Scandium, Titanium, Vanadium, Chromium, Manganese, Iron, Cobalt, Nickel, Copper, Zinc, Yttrium, Zirconium, Niobium, Molybdenum, Technetium, Ruthenium, Rhodium, Palladium, Silver, Cadmium, Hafnium, Tantalum, Tungsten, Rhenium, Osmium, Iridium, Platinum, Gold and Mercury.

By “apoprotein” herein is meant a metalloprotein that has at least one functional metal ion absent or removed. In this context, “functional metal ion” is a metal ion whose absence from the metalloprotein causes either a decrease or elimination of activity. In the case where the metalloprotein has more than one functional metal ion, some embodiments utilize partially demetalated proteins, as long as the loss of some measurable bioactivity is seen with the partially demetalated protein. (Note that “demetalated” and “unmetalated” are used synonymously in most cases herein). In the case where the metalloprotein contains two or more different functional metal ions, “apoprotein” includes the loss of one type of metal ion but not the other, as well as the loss of both. In general, the methods of the invention utilize fully demetalated proteins. It should be noted that in some cases, complete demetalation is not required; for example, even if some of the proteins in the sample retain metal, and thus activity, as long as a decrease in activity is measurable as additional functional metals are removed, the sample is appropriate for use in the methods outlined herein. Suitable apoproteins for use in the invention include all of the apo forms of the metalloproteins listed above.

In some embodiments, the apoproteins are fusion proteins. For example, apoproteins may be modified in a way to form chimeric molecules comprising an apoprotein fused to another, heterologous polypeptide or amino acid sequence. In one embodiment, such a chimeric molecule comprises a fusion of an apoprotein with a tag polypeptide which provides an epitope to which an anti-tag antibody can selectively bind. The epitope tag is generally placed at the amino- or carboxyl-terminus of the apoprotein. The presence of such epitope-tagged forms of an apoprotein can be detected using an antibody against the tag polypeptide. Also, provision of the epitope tag enables the apoprotein polypeptide to be readily purified by affinity purification using an anti-tag antibody or another type of affinity matrix that binds to the epitope tag. These epitope tags can be used for immobilization to a solid support, as outlined herein.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

Other suitable fusion partners include other immobilization components, such as histidine tags for attachment to surfaces with nickel, functional components for the attachment of linkers and labels, etc., and proteinaceous labels. In general, attachment will generally be done as is known in the art, and will depend on the composition of the two materials to be attached. In general, attachment linkers are utilized through the use of functional groups on each component that can then be used for attachment. Preferred functional groups for attachment are amino groups, carboxy groups, oxo groups, hydroxyl groups and thiol groups. These functional groups can then be attached, either directly or indirectly through the use of a linker. Linkers are well known in the art; for example, homo- or hetero-bifunctional linkers as are well known (see 1994 Pierce Chemical Company catalog, technical section on cross-linkers, pages 155-200, incorporated herein by reference). Preferred attachment linkers include, but are not limited to, alkyl groups (including substituted alkyl groups and alkyl groups containing heteroatom moieties), with short alkyl groups, esters, amide, amine, epoxy groups and ethylene glycol and derivatives being preferred.

In one embodiment, particularly when the candidate agents are immobilized on a solid support, a suitable fusion partner is an autofluorescent protein label. Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references are expressly incorporated herein by reference.

In some embodiments, the apoproteins are variants, including amino acid substitutions and deletions, such as truncations. For example, due to the general difficulty of handling transmembrane proteins, either the intracellular domain, or the extracellular domain, depending on the activity to be modulated, may be used (e.g. deletion of the transmembrane domain).

There are a wide number of suitable methods for the generation of apoproteins. In some cases, synthetic methods can be done in the absence of metal ligands, as are described below. This can result in the chemical synthesis of the apoproteins. Protein synthesis methods are well known in the art.

Additionally, recombinant techniques can be used, as outlined below, followed by removal of the metal ions as described below. In general, metalloprotein genes are isolated as is known in the art, generally by using primers and amplification systems such as polymerase chain reaction (PCR) from known sequences such as those found in GenBank. A variety of expression vectors can be made. The expression vectors may be either self-replicating extrachromosomal vectors or vectors which integrate into a host genome. Generally, these expression vectors include transcriptional and translational regulatory nucleic acid operably linked to the nucleic acid encoding the metalloprotein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the metalloprotein; for example, transcriptional and translational regulatory nucleic acid sequences from Bacillus are preferably used to express the metalloprotein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

The metalloproteins of the present invention are produced by culturing a host cell transformed with an expression vector containing nucleic acid encoding a metalloprotein, under the appropriate conditions to induce or cause expression of the metalloprotein. The conditions appropriate for metalloprotein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archebacteria, fungi, and insect and animal cells, including mammalian cells. Of particular interest are Drosophila melangaster cells, Saccharomyces cerevisiae and other yeasts, E. coli, Bacillus subtilis, SF9 cells, C129 cells, 293 cells, Neurospora, BHK, CHO, COS, and HeLa cells, fibroblasts, Schwanoma cell lines, and other immortalized mammalian cell lines.

In a preferred embodiment, the metalloproteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for Metalloprotein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, metalloproteins are expressed in bacterial systems. Bacterial expression systems are well known in the art. A suitable bacterial promoter is any nucleic acid sequence capable of binding bacterial RNA polymerase and initiating the downstream (3′) transcription of the coding sequence of the metalloprotein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the metalloprotein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

These components are assembled into expression vectors. Expression vectors for bacteria are well known in the art, and include vectors for Bacillus subtilis, E. coli, Streptococcus cremoris, and Streptococcus lividans, among others.

The bacterial expression vectors are transformed into bacterial host cells using techniques well known in the art, such as calcium chloride treatment, electroporation, and others.

In one embodiment, metalloproteins are produced in insect cells. Expression vectors for the transformation of insect cells, and in particular, baculovirus-based expression vectors, are well known in the art.

In a preferred embodiment, the metalloprotein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

In a preferred embodiment, the metalloprotein is purified or isolated after expression. Metalloproteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the metalloprotein may be purified using a standard anti-metalloprotein antibody column. Ultrafiltration and diafiltration techniques, in conjunction with protein concentration, are also useful. For general guidance in suitable purification techniques, see Scopes, R., Protein Purification, Springer-Verlag, N.Y. (1982). The degree of purification necessary will vary depending on the use of the metalloprotein. In some instances no purification will be necessary.

In some embodiments, the metalloproteins are made with the metal present, and thus the metalloprotein is generally demetalated to form the apoprotein form. In general, this is accomplished in several conceptual steps. The formal steps include a) destablization of the protein fold by additives that alter solution pH, ionic strength, solvent composition, reducing agent concentration or detergent/denaturant concentration, b) addition of metal-binding agents such as chelating agents, described below, that bind to the metal of interest, and c) removal of additives by dialysis, exchange or gel filtration. These formal steps are typically executed consecutively, but for proteins with weakly bound metals, some steps are simultaneous.

Suitable metal-binding agents include, but are not limited to, nitrilotriacetic acid (NTA), EDTA, EGTA, bipyridine, acetonitrile, orthophenanthroline, bathocuproine sulfonate (BCS), Neocuproine, ethylenediamine, dithiolthreitol (DTT) and TPEN. In some embodiments, the metal binding agent need not be a chelator, for instance mercaptoethanol, thiocyanide, pyridine, and cyanide ion can be used in the second step to remove the metal. The metal removing agents may be solution phase compounds or may be attached to polymer supports (ie Chelex™). Preferred reducing agents include, but are not limited to mercaptoethanol, dithiothreitol, ascorbic acid, and sodium borohydride. Preferred detergents and denaturants include, but are not limited to sodium dodecylsulfonate (SDS), guanidium hydrochloride, and urea. For example when a copper metalloprotein is to be used, it is demetallated by first lowering the solution pH to a point above the pI of the protein in the presence of the Cu(II) reducing agent sodium ascorbate, adding the copper binding agent bathcuproine sulfonate and incubating for 8 hrs at 4C. One the metal ion is removed in this manner, the solution is dialyzed to remove the additives and exchange the solution with the assay buffer.

In some metalloproteins, the presence of disulfides results in the resistance of the protein to demetalation. In these cases, reducing the disulfide prior to, or simultaneous with, the addition of chelator can be done. Special buffer and atmosphere considerations are required. For instance, acidic pH will decrease the reduction potential of the additive and should be avoided for this case. These steps should be conducted under an inert atmosphere such as N2 or Ar in order to avoid reoxidation of the thiols by dioxygen. Preferred disulfide reducing agents include DTT, mercaptoethanol and TCEP.

In some embodiments, the metalloproteins are made as apoproteins. This can be accomplished in several ways. In one embodiment, the metalloproteins are synthesized using protein synthetic methods in the absence of the relevant metal ions. As will be appreciated by those in the art, care is taken to ensure no contamination of the protein with metal.

In some cases, for example when the metalloprotein is expressed in a heterologous system (for example E. coli for non-E. coli metalloproteins) the proteins may effectively be made as apoproteins, due to the high expression levels and/or the purification methods used. For example, high expression levels may result in the metalation pathways being overwhelmed such that apoproteins are produced. Similarly, proteins made as inclusion bodies may not contain metal, and/or the denaturation/renaturation process results in apoprotein forms (particularly if chelators are included in the buffer system).

Once expressed, purified and demetalated, if necessary, the apoproteins are useful in a number of applications, and in particular, screening assays for the identification of candidate agents that bind to the apoproteins and prevent metalation, such that bioactivity is decreased.

Screens may be designed to first find candidate agents that can bind to metalloproteins, and then these agents may be used in assays that evaluate the ability of the candidate agent to modulate bioactivity. Thus, as will be appreciated by those in the art, there are a number of different assays which may be run; binding assays and activity assays.

Thus, in a preferred embodiment, the methods comprise combining a metalloprotein and a candidate bioactive agent, and determining the binding of the candidate agent to the metalloprotein.

### Assay Components

In general, as described herein, the assays are done by contacting an apoprotein with one or more candidate agents to be tested. By “candidate agent” or “candidate drug” as used herein describes any molecule, e.g., proteins including biotherapeutics including antibodies and enzymes, small organic molecules including known drugs and drug candidates, polysaccharides, fatty acids, vaccines, nucleic acids, etc. that can be screened for activity as outlined herein. Candidate agents are evaluated in the present invention for discovering potential therapeutic agents that affect activity and therefore potential disease states, for elucidating toxic effects of agents (e.g. environmental pollutants including industrial chemicals, pesticides, herbicides, etc.), drugs and drug candidates, food additives, cosmetics, etc., as well as for elucidating new pathways associated with agents (e.g. research into the side effects of drugs, etc.).

Candidate agents encompass numerous chemical classes. In one embodiment, the candidate agent is an organic molecule, preferably small organic compounds having a molecular weight of more than 100 and less than about 2,500 daltons. Particularly preferred are small organic compounds having a molecular weight of more than 100 and less than about 2,000 daltons, more preferably less than about 1500 daltons, more preferably less than about 1000 daltons, more preferably less than 500 daltons. Candidate agents comprise functional groups necessary for structural interaction with proteins, particularly hydrogen bonding, and typically include at least one of an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

“Known drugs” or “known drug agents” or “already-approved drugs” refers to agents (i.e., chemical entities or biological factors) that have been approved for therapeutic use as drugs in human beings or animals in the United States or other jurisdictions. In the context of the present invention, the term “already-approved drug” means a drug having approval for an indication distinct from an indication being tested for by use of the methods disclosed herein.

Candidate agents are obtained from a wide variety of sources including libraries of synthetic or natural compounds. For example, numerous means are available for random and directed synthesis of a wide variety of organic compounds and biomolecules, including expression and/or synthesis of randomized oligonucleotides and peptides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or (L)-configuration. If non-naturally occurring side chains are used, non-amino acid substituents may be used, for example to prevent or retard in vivo degradations. Peptide inhibitors of enzymes find particular use.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the systems described herein. Particularly preferred in this embodiment are libraries of bacterial, fungal, viral, and mammalian proteins, with the latter being preferred, and human proteins being especially preferred.

In some embodiments, the candidate agents are peptides. In this embodiment, it can be useful to use peptide constructs that include a presentation structure. By “presentation structure” or grammatical equivalents herein is meant a sequence, which, when fused to candidate bioactive agents, causes the candidate agents to assume a conformationally restricted form. Proteins interact with each other largely through conformationally constrained domains. Although small peptides with freely rotating amino and carboxyl termini can have potent functions as is known in the art, the conversion of such peptide structures into pharmacologic agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems. A number of workers have constructed small domain molecules in which one might present randomized peptide structures. Preferred presentation structures maximize accessibility to the peptide by presenting it on an exterior loop. Accordingly, suitable presentation structures include, but are not limited to, minibody structures, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc. See U.S. Pat. No. 6,153,380, incorporated by reference.

Of particular use in screening assays are phage display libraries; see e.g., U.S. Pat. Nos. 5,223,409; 5,403,484; 5,571,698; and 5,837,500, all of which are expressly incorporated by reference in their entirety for phage display methods and constructs.

In a preferred embodiment, the candidate agents are antibodies, a class of proteins. The term “antibody” includes full-length as well antibody fragments, as are known in the art, including Fab Fab2, single chain antibodies (Fv for example), chimeric antibodies, humanized and human antibodies, etc., either produced by the modification of whole antibodies or those synthesized de novo using recombinant DNA technologies, and derivatives thereof.

In a preferred embodiment, the candidate bioactive agents are nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), all of which are incorporated by reference)). Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleoside & Nucleotide, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook, and peptide nucleic acids. Nucleic acids containing one or more carbocyclic sugars are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, 4-acetylcytosine, 8-hydroxy-N-6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. etc.

In one embodiment, the nucleic acids are aptamers, see U.S. Pat. Nos. 5,270,163, 5,475,096, 5,567,588, 5,595,877, 5,637,459, 5,683,867, 5,705,337, and related patents, hereby incorporated by reference.

It should be noted in the context of the invention that nucleosides (ribose plus base) and nucleotides (ribose, base and at least one phosphate) are used interchangeably herein unless otherwise noted.

As described above generally for proteins, nucleic acid candidate bioactive agents may be naturally occurring nucleic acids, random and/or synthetic nucleic acids. For example, digests of procaryotic or eucaryotic genomes may be used as is outlined above for proteins. In addition, RNAis are included herein.

“Food additive” includes, but is not limited to, organoleptic agents (i.e., those agents conferring flavor, texture, aroma, and color), preservatives such as nitrosamines, nitrosamides, N-nitroso substances and the like, congealants, emulsifiers, dispersants, fumigants, humectants, oxidizing and reducing agents, propellants, sequestrants, solvents, surface-acting agents, surface-finishing agents, synergists, pesticides, chlorinated organic compounds, any chemical ingested by a food animal or taken up by a food plant, and any chemical leaching into (or otherwise finding its way into) food or drink from packaging material. The term is meant to encompass those chemicals which are added into food or drink products at some step in the manufacturing and packaging process, or find their way into food by ingestion by food animals or uptake by food plants, or through microbial byproducts such as endotoxins and exotoxins (pre-formed toxins such as botulinin toxin or aflatoxin), or through the cooking process (such as heterocyclic amines, e.g., 2-amino-3-methyllimidazo[4,5-f]quinolone), or by leaching or some other process from packaging material during manufacturing, packaging, storage, and handling activities.

“Industrial chemical” includes, but is not limited to, volatile organic compounds, semi-volatile organic compounds, cleaners, solvents, thinners, mixers, metallic compounds, metals, organometals, metalloids, substituted and non-substituted aliphatic and acyclic hydrocarbons such as hexane, substituted and non-substituted aromatic hydrocarbons such as benzene and styrene, halogenated hydrocarbons such as vinyl chloride, aminoderivatives and nitroderivatives such as nitrobenzene, glycols and derivatives such as propylene glycol, ketones such as cyclohexanone, aldehydes such as furfural, amides and anhydrides such as acrylamide, phenols, cyanides and nitrites, isocyanates, and pesticides, herbicides, rodenticides, and fungicides.

“Environmental pollutant” includes any chemical not found in nature or chemicals that are found in nature but artificially concentrated to levels exceeding those found in nature (at least found in accessible media in nature). So, for example, environmental pollutants can include any of the non-natural chemicals identified as an occupational or industrial chemical yet found in a non-occupational or industrial setting such as a park, school, or playground. Alternatively, environmental pollutants may comprise naturally occurring chemicals such as lead but at levels exceeding background (for example, lead found in the soil along highways deposited by the exhaust from the burning of leaded gasoline in automobiles). Environmental pollutants may be from a point source such as a factory smokestack or industrial liquid discharge into surface or groundwater, or from a non-point source such as the exhaust from cars traveling along a highway, the diesel exhaust (and all that it contains) from buses traveling along city streets, or pesticides deposited in soil from airborne dust originating in farmlands. As used herein, “environmental contaminant” is synonymous with “environmental pollutant.”

In general, libraries of candidate agents are tested. By “library” herein is meant a plurality of molecules, and in general is at least 10² to 10⁸ molecules, with from about 10² to about 10⁴ to 10⁶ being preferred.

The candidate agents are contacted with the apoprotein under reaction conditions that favor agent-target interactions. Generally, this will be physiological conditions. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away, in the case of solid phase assays. Assay formats are discussed below.

A variety of other reagents may be included in the assays. These include reagents like salts, neutral proteins, e.g. albumin, detergents, etc which may be used to facilitate optimal apoprotein-agent binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

In one embodiment, solution phase binding assays are done in the absence of metal ions (solution phase assays using metalloprotein-ligand complexs are outlined below). Generally in this embodiment, fluorescence resonance energy transfer (FRET) assays are done, by labeling both the candidate agents and apoproteins with different fluorophores with overlapping spectra. As energy transfer is distance dependent, in the absence of binding the excitation at one wavelength does not produce an emission spectra. Only if the two labels are close, e.g. when binding has occurred, will excitation at one wavelength result in the desired emission spectra of the second label.

In some embodiments, solid phase (heterogeneous) assays are done. In this case, binding assays are done wherein either the metalloprotein or the candidate agent is non-diffusably bound to an insoluble solid support, and detection is done by adding the other component which is labeled, as described below.

The insoluble supports may be made of any composition to which the compositions can be bound, is readily separated from soluble material, and is otherwise compatible with the overall method of screening. The surface of such supports may be solid or porous and of any convenient shape. Examples of suitable supports include microtiter plates, arrays, membranes and beads, and include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica based materials including silicon and modified silicon, carbon, metals, inorganic glasses, plastics, ceramics, and a variety of other polymers. In a some embodiments, the solid supports allow optical detection and do not themselves appreciably fluoresce. In addition, as is known the art, the solid support may be coated with any number of materials, including polymers, such as dextrans, acrylamides, gelatins, agarose, etc. Exemplary solid supports include silicon, glass, polystyrene and other plastics and acrylics. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable.

In a preferred embodiment, the metalloprotein (either attached prior to demetalation or after) is bound to the support, and a library of candidate bioactive agents are added to the assay. Alternatively, the candidate agent is bound to the support and the metalloprotein is added. Attachment to the solid support is accomplished using well known methods, and will depend on the composition of the two materials to be attached. In general, for covalent attachment, attachment linkers are utilized through the use of functional groups on each component that can then be used for attachment. Preferred functional groups for attachment are amino groups, carboxy groups, oxo groups, hydroxyl groups and thiol groups. These functional groups can then be attached, either directly or indirectly through the use of a linker. Linkers are well known in the art; for example, homo- or hetero-bifunctional linkers as are well known (see 1994 Pierce Chemical Company catalog, technical section on cross-linkers, pages 155-200, incorporated herein by reference). In some embodiments, absorption or ionic interactions are utilized. In some cases, small molecule candidate agents are synthesized directly on microspheres, for example, which can then be used in the assays of the invention.

Following binding of the protein or agent, excess unbound material is removed by washing. The surface may then be blocked through incubation with bovine serum albumin (BSA), casein or other innocuous protein or other moiety.

In the binding assays, either the metalloprotein, the candidate agent (or, in some cases, the metal binding component, described below) is labeled. By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

Specific labels include optical dyes, including, but not limited to, chromophores, phosphors and fluorophores, with the latter being specific in many instances. Fluorophores can be either “small molecule” fluores, or proteinaceous fluores as described above. The labeled metal donor (e.g. the metal binding component) can be a chemical probe (such as Zinquin or Zinbo5) which undergoes a spectroscopic change when it releases the metal ion as described herein.

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland, hereby expressly incorporated by reference.

In one embodiment, the apoprotein is attached to the support, adding labeled candidate agents, washing off excess reagent, and determining whether the label is present on the solid support. Various blocking and washing steps may be utilized as is known in the art.

In one embodiment, the candidate agents are immobilized to the support, and a labeled apoprotein is added to determine binding.

In some embodiments, binding is determined by the addition of a metal (in the form of a metal-binding agent, described below). The metalation of the apoprotein can be followed in a variety of ways, as described herein. In one embodiment, metal-binding agents are used that are fluorescent when the metal is associated with the agent; upon addition to the apoprotein, the transfer of metal from the metal-binding agent to the metalloprotein results in a loss of fluorescence of the mixture. Similarly, an increase in fluorescence can be seen when the metal in the metal-binding agent is quenching the intrinsic fluorescence of the metal-binding agent. In some embodiments, ratiometric analysis can be done, where fluorescence of the mixture is measured at least two different wavelengths.

Alternatively, the presence or absence of metalation is tested optically; for example, some metalloproteins alter their absorbance spectra upon addition or loss of the metal. For example, some proteins are different spectroscopic changes (for instance colors) depending on the presence or absence of a metal ion. Similarly, conformation changes can be monitored using UV/Visible absorbance or fluorescence. For example, the lack of structural metal ions can “loosen” the conformation structure such that the absorbance or fluorescence of tryptophan and tyrosine are altered, relative to the structure with metal present.

Accordingly, some assays utilize the addition of metal-binding agents to the mixture. By “metal-binding agent” herein is meant a complex comprising a metal ion, as defined herein, and a metal binding component. This can also be referred to herein as a “metal donating agent”, in the case where the complex is used to add metal for bioactivity. In general, the metals in the metal-ligand complexes are metal ions, also referred to herein as “metals”.

By “metal binding component” herein is meant a compound that provides a coordinate covalent bond. The term in this context includes counterions, “traditional” metal-binding molecules including ligands and chelators, as well as other metalloproteins Standard counterions include, but are not limited to, Cl, Cl2, SO4, carbonate, etc.

As will be appreciated by those in the art, a wide variety of suitable chelators can be used. In general, the chelator has a number of coordination sites containing coordination atoms which bind the metal ion. The number of coordination sites is an intrinsic characteristic of the chelating agent: those molecules that use two different atoms to form two bonds to a metal are said to be bidentate. The terms tridentate, tetradentate, pentadentate, etc then refer to chelating agents that use three, four and five atoms to form the same number of bonds respectively. In general, the metalloprotein-ligand complex should have a Kd that allows for the removal of the metal ion from the metalloprotein-ligand complex for binding to the apoprotein.

In some embodiments, the chelators can be macrocyclic or non-macrocyclic in structure. Suitable chelators include, but are not limited to, 4-aminopyridine; 4-dimethylpyridine; 4-acetylpyridine; 4-nitropyridine; 4,4′-diamino-2,2′-bipyridine; 5,5′-diamino-2,2′-bipyridine; 6,6′-diamino-2,2′-bipyridine; 5,5′-dimethyl-2,2′-bipyridine; 6,6′-dimethyl-2,2′-bipyridine; 4,4′-diethylenediamine-2,2′-bipyridine; 5,5′-diethylenediamine-2,2′-bipyridine; 6,6′-diethylenediamine-2,2′-bipyridine; 4,4′-dihydroxyl-2,2′-bipyridine; 5,5′-dihydroxyl-2,2′-bipyridine; 6,6′-dihydroxyl-2,2′-bipyridine; 4,4′,4″-triamino-2,2′,2″-terpyridine; 4,4′,4″-triethylenediamine-2,2′,2″-terpyridine; 4,4′,4″-trihydroxy-2,2′,2″-terpyridine; 4,4′,4″-trinito-2,2′,2″-terpyridine; 4,4′,4″-triphenyl-2,2′,2″-terpyridine; 4,7-diamino-1,10-phenanthroline; 3,8-diamino-1,10-phenanthroline; 4,7-diethylenediamine1,10-phenanthroline; 3,8-diethylenediamine-1,10-phenanthroline; 4,7-dihydroxyl-1,10-phenanthroline; 3,8-dihydroxyl-1,10-phenanthroline; 4,7-dinitro-1,10-phenanthroline; 3,8-dinitro-1,10-phenanthroline; 4,7-diphenyl-1,10-phenanthroline; 3,8-diphenyl-1,10-phenanthroline; 4,7-disperamine-1,10-phenanthroline; 3,8-disperamine-1,10-phenanthroline; dipyrido[3,2-a:2′,2′-c]phenazine; 4,4′-dichloro-2,2′-bipyridine; 5,5′-dichloro-2,2′-bipyridine; and 6,6′-dichloro-2,2′-bipyridine, picolinic acid, etc.

In some embodiments, the metalloprotein-ligand complexes have optical properties that are different depending on the presence or absence of the metal. For example, there are complexes of Zn that are fluorescent when the Zn is part of the metalloprotein-ligand complex, but upon removal of the Zn, the remaining chelator is not fluorescent. Thus, upon incubation of the metalloprotein-ligand complex with the apoprotein, the Zn is transferred to the apoprotein to reconstitute the metalloprotein and is accompanied by a decrease in fluorescence. That is, in some cases, dyes can be used to determine metalation status by competition methods. For example, for Zn metalloproteins, the many types of metal-specific fluorescent probes, such as those in the Zinbo and Zinquin families can be used. These probes are fluorescent dyes coupled to Zn chelators that alter their absorption or emission spectra upon Zn binding (see Taki et al, JACS 126:712 (2994) and Fahrni et al., JACS 121:11448 (1999), both of which are hereby incorporated by reference in their entirety). If a candidate agent blocks Zn-acquisition by a target protein in the assay after a given incubation time, then the subsequent aliquot of the Zn-probe complex will not undergo a change in fluorescence. In control assays, those compounds that do not block acquisition of Zn the target protein will allow the target apo-mp to take Zn from the Zn-probe complex and a change in the fluorescence of the sample will be observed.

In some embodiments, the metalloprotein-ligand complex is a metalloprotein. In this embodiment, the metalloprotein chosen preferably has a lower affinity for the metal than the apoprotein for which it serves as the metalloprotein-ligand complex, to allow transfer of the metal ion to the apoprotein if inhibition by a candidate agent has not occurred. In some cases, the metalloprotein-ligand complex is a metallochaperone protein that is known to facilitate metalation of a particular apoprotein. Thus, for example, there are pairs of apoproteins-metal chaperones that find use in the invention. Suitable pairs include, but are not limited to, superoxide dismutase and the Copper Chaperone for SOD1 (known as CCS. For instance the copper chaperone Atx1 binds Cu(I) on the surface and can transfer this metal to other proteins including the p-type ATPases7a, 7b and CCC2. Other proteins that are proposed to serve as metal donors in living systems include metallothionein, serum albumin, PcoC and Frataxin. These proteins can be employed as metal-donors in the reconstitution step of the screening assays.

The addition of the metal-ligand complex can be done simultaneously with the addition of the candidate agents to the apoprotein, or afterwards, with the latter being preferred.

One the components (apoprotein, candidate agent and metalloprotein-ligand complex) have be mixed, the detection of the presence of metal ions in the protein occurs, which is an indication of binding of the candidate agent and its ability to interfere with metalation. As outlined herein, this can be done in a variety of ways, generally by spectroscopic, including fluorescence, methods.

In some embodiments, bioactivity assays are done to test whether the candidate agent has inhibited metalation of the apoprotein and thus inhibited bioactivity. As for binding assays, activity assays can be either solution based, or rely on the use of components that are immobilized on solid supports. In this case, the bioactivity assay depends on the bioactivity of the metalloprotein, and will be run accordingly. Thus, for example, metalloenzyme activity assays are well known, using a wide variety of generally commercially available substrates. In this application, the ability of a give agent to prevent recovery of native enzyme activity upon addition of a metal ion to the apo-protein/candidate compound mixture will be scored. This method provides a route to identify candidate agents that are capable of altering the target protein in a manner that prevents metal binding.

Similarly, activity assays based on metalloprotein activity, such as transcription factor activation of gene expression, can be done.

In one embodiment, any of the assays outlined herein can utilize robotic systems for high throughput screening. Many systems are generally directed to the use of 96 (or more) well microtiter plates, but as will be appreciated by those in the art, any number of different plates or configurations may be used. In addition, any or all of the steps outlined herein may be automated; thus, for example, the systems may be completely or partially automated.

As will be appreciated by those in the art, there are a wide variety of components which may be used, including, but not limited to, one or more robotic arms; plate handlers for the positioning of microplates; automated lid handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In a preferred embodiment, chemically derivatized particles, plates, tubes, magnetic particle, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In a preferred embodiment, platforms for multi-well plates, multi-tubes, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, electroporator, and multi-position work decks for source samples, sample and reagent dilution, assay plates, sample and reagent reservoirs, pipette tips, and an active wash station.

In a preferred embodiment, thermocycler and thermoregulating systems are used for stabilizing the temperature of the heat exchangers such as controlled blocks or platforms to provide accurate temperature control of incubating samples from 4° C. to 100° C.

In some preferred embodiments, the instrumentation will include a detector, which may be a wide variety of different detectors, depending on the labels and assay. In a preferred embodiment, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), SPR systems, luminescence, quenching, two-photon excitation, and intensity redistribution; CCD cameras to capture and transform data and images into quantifiable formats; and a computer workstation. These will enable the monitoring of the size, growth and phenotypic expression of specific markers on cells, tissues, and organisms; target validation; lead optimization; data analysis, mining, organization, and integration of the high-throughput screens with the public and proprietary databases.

These instruments can fit in a sterile laminar flow or fume hood, or are enclosed, self-contained systems as needed. Flow cytometry or capillary electrophoresis formats may be used for individual capture of magnetic and other beads, particles, cells, and organisms.

The flexible hardware and software allow instrument adaptability for multiple applications. The software program modules allow creation, modification, and running of methods. The system diagnostic modules allow instrument alignment, correct connections, and motor operations. The customized tools, labware, and liquid, particle, cell and organism transfer patterns allow different applications to be performed. The database allows method and parameter storage. Robotic and computer interfaces allow communication between instruments.

In a preferred embodiment, the robotic workstation includes one or more heating or cooling components. Depending on the reactions and reagents, either cooling or heating may be required, which may be done using any number of known heating and cooling systems, including Peltier systems.

In a preferred embodiment, the robotic apparatus includes a central processing unit that communicates with a memory and a set of input/output devices (e.g., keyboard, mouse, monitor, printer, etc.) through a bus. The general interaction between a central processing unit, a memory, input/output devices, and a bus is known in the art. Thus, a variety of different procedures, depending on the experiments to be run, are stored in the CPU memory.

In additional embodiments, the invention provides methods of inhibiting the apoprotein form of metalloproteins by contacting the apoprotein with an inhibitor such that metal binding to the apoprotein form is decreased, as compared to the metal binding in the absence of the inhibitor. In this case, “decreased” is generally at least a 5-20-25% decrease in the amount of metal bound per protein, with over 50-75% being useful in some embodiments and a 95-98-100% loss of metal being useful as well. In general, the loss of metal correlates to a loss of biological activity, as measured depending on the metalloprotein, with the same decreases in activity being useful. These methods rely on the fact that there is a larger “pool” of apoproteins that exist in the cytoplasm, prior to metalation, than was previously thought.

In a further embodiment, the invention provides methods of treating a metalloprotein-associated disorder comprising contacting an apoprotein form of the metalloprotein with an inhibitor such that metal binding to the apoprotein form is decreased. In this context, a “metalloprotein-associated disorder” is a disorder either directly or indirectly related to a metalloprotein.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. The references cited herein are expressly incorporated by reference.

1. A method for screening for binding to metalloproteins comprising:
a) providing an apoprotein;
b) contacting said apoprotein with a candidate agent to form a mixture; and
c) determining the binding of said candidate agent to said apoprotein.

2. A method according to claim 1 further comprising:
a) adding a metal ligand complex to said mixture; and
b) determining the bioactivity of said protein.

3. A method according to claim 1 wherein said candidate agent is labeled.

4. A method according to claim 3 wherein said label is a fluorophore.

5. A method according to claim 1 wherein said apoprotein is immobilized on a solid support.

6. A method according to claim 5 wherein said solid support is a bead.

7. A method according to claim 1 wherein said candidate agent is immobilized on a solid support.

8. A method according to claim 7 wherein said apoprotein is labeled.

9. A method according to claim 8 wherein said label is a fluorophore.

10. A method according to claim 9 wherein said fluorophore is a fluorescent protein.

11. A method according to claim 1 further comprising:
a) providing a metalyated protein; and
b) removing the metal to form the apoprotein.

12. A method according to claim 11 wherein said removing step utilizes reducing at least one disulfide bond of said protein.

13. A method according to claim 2 further comprising:
a) identifying at least one candidate drug that modulates bioactivity of said protein;
b) contacting said candidate drug with a cell expressing said protein; and
c) determining the effect of said drug on said cell.

14. A method according to claim 1 wherein a library of candidate agents are added to at least one apoprotein to form a plurality of mixtures.

15. A method according to claim 1 wherein a library of apoproteins are tested.

16. A method of inhibiting the bioactivity of a metalloprotein comprising contacting an apoprotein form of said metalloprotein with an inhibitor such that metal binding to said apoprotein form is decreased.

17. A method of treating a metalloprotein-associated disorder comprising contacting an apoprotein form of said metalloprotein with an inhibitor such that metal binding to said apoprotein form is decreased.

18. A method according to claim 14 wherein a library of apoproteins are tested.
